(19) United States
(12) Patent Application Publication
(10) Pub. No.: US 2025/0269206 A1
(43) Pub. Date: Aug. 28, 2025

(54) TARGET STRUCTURE TRACKING FOR RADIATION THERAPY

(71) Applicant: SIEMENS HEALTHINEERS INTERNATIONAL AG, Steinhausen (CH)

(72) Inventor: Willem VERBAKEL, Amsterdam (NL)

(73) Assignee: SIEMENS HEALTHINEERS INTERNATIONAL AG, Steinhausen (CH)

(21) Appl. No.: 18/586,520

(22) Filed: Feb. 25, 2024

(51) Int. Cl. A61N 5/10; G06T 7/246; G06T 7/73

(52) U.S. Cl. CPC A61N 5/1049; A61N 5/1039; G06T 7/248; G06T 7/74; G06T 2207/10116; G06T 2207/20081; G06T 2207/30096

(57) ABSTRACT

Example methods and systems for target structure tracking for radiation therapy are described. In one example, a computer system may obtain first and second projection image data. The first projection image data may be generated using a first imaging source to emit a first imaging beam towards an imager to image a target structure from a first angle. The second projection image data may be generated using a second imaging source to emit a second imaging beam towards the imager to image the target structure from a second angle. The computer system may process the first projection image data to determine first two-dimensional (2D) position data and the second projection image data to determine second 2D position data. Based on at least the first and second 2D position data, the computer system may determine three-dimensional (3D) position data associated with the target structure, thereby tracking the target structure in 3D.

### BACKGROUND

Radiation therapy is a widely used cancer treatment modality that uses high-energy radiation to reduce or eliminate cancerous tumors. In practice, applied radiation does not inherently discriminate between a tumor and proximal healthy structures, such as organs, etc. Ideally, the objective is to deliver a lethal or curative radiation dose to the tumor, while maintaining an acceptable dose level in the proximal healthy structures. During treatment time, one of the important factors for effective delivery of radiation treatment is the location of the target structure inside a planned target volume (PTV), which is the volume where high dose of radiation is delivered. Due to intrafraction motion of the patient, or the tumor, the tumor could move outside this PTV during treatment. A system based on repeated kiloVoltage (kV) imaging and so-called “tracking” software may be used to monitor the position of the tumor or any other suitable target structure. In practice, however, there may be various challenges associated with target structure tracking (also referred to as position monitoring) during treatment.

### BRIEF DESCRIPTION OF DRAWINGS

FIG. 1 is a schematic diagram illustrating an example radiation therapy system having a ring-based configuration to facilitate target structure tracking for radiation therapy;

FIG. 2A is a schematic diagram illustrating a first example configuration of an imaging system shown in FIG. 1;

FIG. 2B is a schematic diagram illustrating a second example configuration of an imaging system shown in FIG. 1;

FIG. 3 is a schematic diagram illustrating an example process for a computer system to perform target structure tracking for radiation therapy;

FIG. 4 is a flowchart of a first example detailed process for treatment planning and delivery;

FIG. 5A is a schematic diagram illustrating a first example of imaging beam generation using multiple imaging sources and an imager;

FIG. 5B is a schematic diagram illustrating a second example of imaging beam generation using multiple imaging sources and an imager;

FIG. 6 is a flowchart of an example process for two-dimensional (2D) position data estimation;

FIG. 7 is a flowchart of a second example detailed process for treatment planning and delivery;

FIG. 8A is a schematic diagram illustrating a first example of three-dimensional (3D) position data estimation using an artificial intelligence (AI) engine;

FIG. 8B is a schematic diagram illustrating a second example of 3D position data estimation using an AI engine; and

FIG. 9 is a schematic diagram illustrating an example radiation therapy system having a C-arm configuration to facilitate target structure tracking for radiation therapy.

### SUMMARY

According to examples of the present disclosure, target structure tracking for radiation therapy may be performed in an improved manner. In one example, multiple imaging sources and an imager may be used to image a target structure within a patient from different angles, either substantially simultaneously or sequentially. This in turn facilitates two-dimensional (2D) tracking as well as three-dimensional (3D) tracking of the target structure. As used herein, the term “target structure tracking” may refer generally to estimating position data associated with a target structure, such as to facilitate position monitoring and/or verification, target localization or the like during radiation treatment. The term “target structure” may refer generally to any suitable structure that requires tracking, such as tumor, organ-at-risk (OAR), healthy tissue, bony structure (e.g., vertebra), implanted marker, brachytherapy applicator for brachytherapy, etc.

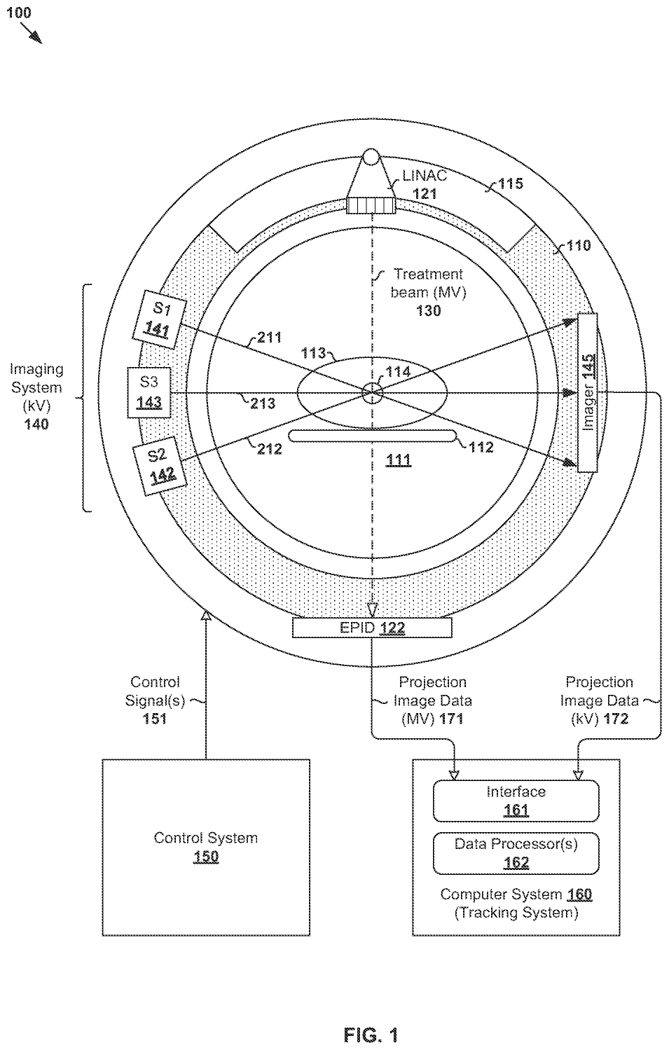

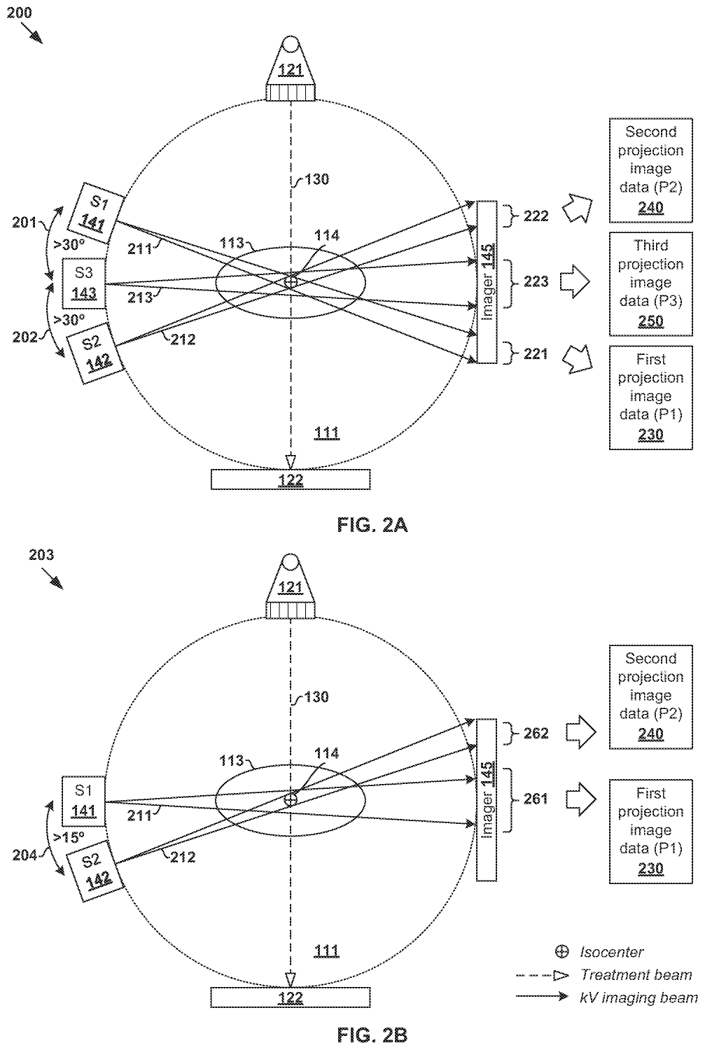

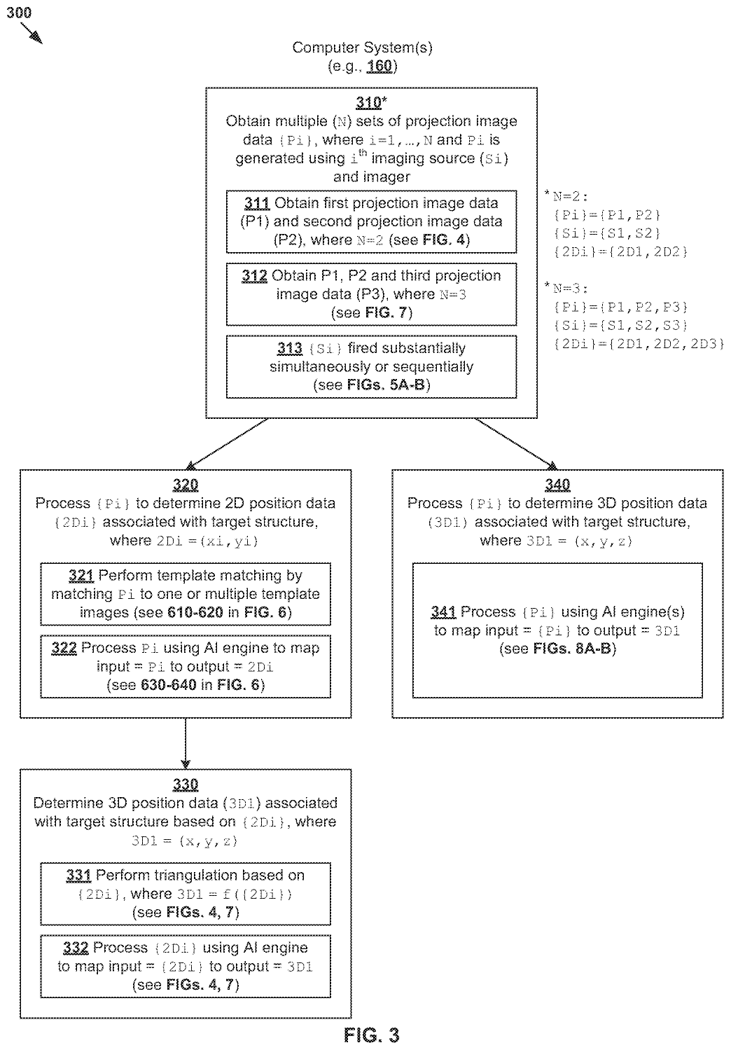

According to a first aspect, examples of the present disclosure provide method(s) and computer system(s) for target structure tracking. In one example, a computer system (see 160 in FIG. 1) may obtain multiple sets of projection image data that include first projection image data and second projection image data (e.g., 230-240 in FIGS. 2A-B) using multiple imaging sources and an imager of a radiation therapy system. The first projection image data may be generated using a first imaging source to emit a first imaging beam towards a first imaging area on the imager to image a target structure from a first angle. The second projection image data may be generated using a second imaging source to emit a second imaging beam towards a second imaging area on the same imager to image the target structure from a second angle. The computer system may process (a) the first projection image data to determine first 2D position data associated with the target structure and (b) the second projection image data to determine second 2D position data associated with the target structure. Based on at least the first 2D position data and second 2D position data, the computer system may determine three-dimensional (3D) position data associated with the target structure, thereby tracking the target structure in 3D. See also 310-330 in FIG. 3.

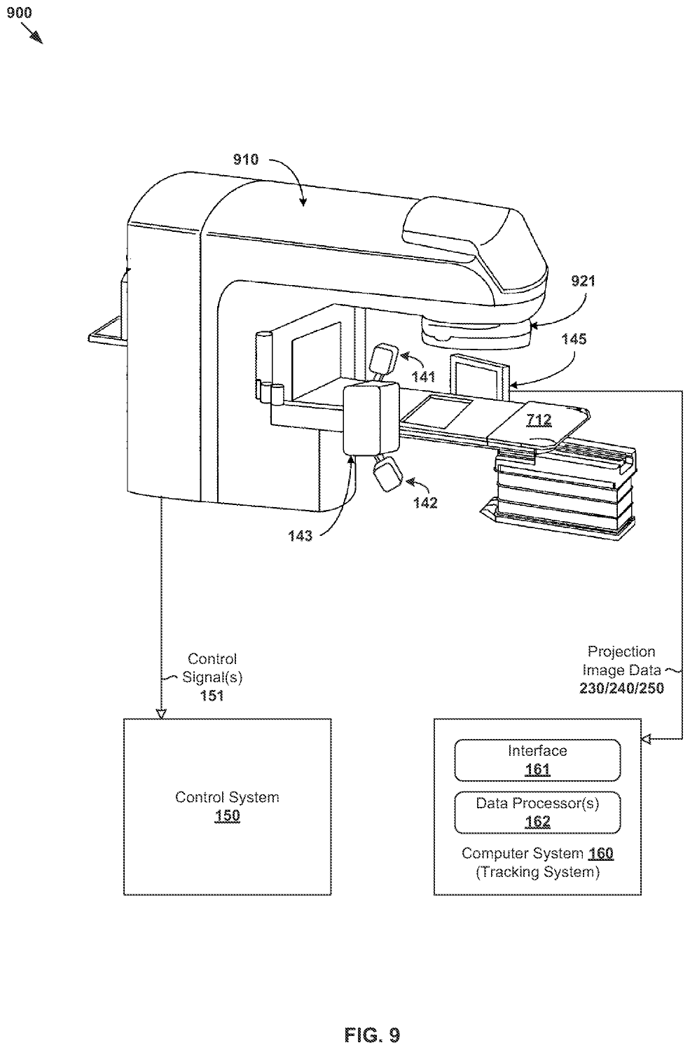

According to a second aspect, examples of the present disclosure provide radiation therapy system(s) for target structure tracking. In one example, a radiation therapy system (see FIG. 1 and FIG. 9) may include an imager and multiple imaging sources, such as a first imaging source and a second imaging source. The first imaging source may be configured to emit a first imaging beam towards a first imaging area on the imager to image a target structure within a patient from a first angle, thereby generating first projection image data. The second imaging source may be configured to emit a second imaging beam towards a second imaging area on the imager to image the target structure from a second angle, thereby generating second projection image data. The radiation therapy system may further include a computer system to (a) obtain the first projection image data and the second projection image data and (b) determine 3D position data associated with the target structure based on the first projection image data and the second projection image data. See also 310, 340 in FIG. 3.

Examples of the present disclosure should be contrasted against conventional approaches that rely on a single imaging source and a single detector for imaging patients. In these conventional approaches, 3D position data estimation may involve template matching and triangulation with earlier matches (≥20 degrees earlier). In practice, such conventional approaches might have a few issues. First, before the first 3D position could be determined, 2D matches of an angle of ≥20 degrees are required for triangulation. As such, there is usually a time lag between the start of irradiation and when the first 3D position is available. Due to the high doses given, gantry speed may slow down to 1 degree per second during irradiation, causing a longer time lag before triangulation starts.

Second, triangulation with earlier data may lead to inaccurate 3D position estimation, especially when the target structure has moved in the time between two images are taken. Third, when an image has low contrast or high noise at certain gantry angles (e.g., due to over projection of shoulders or arms, etc.), template matching that involves normalized cross correlation may lead to the wrong results. These wrong 2D matches may then reduce the accuracy of subsequent 3D position data estimation. In at least some embodiments, target structure tracking according to the examples of the present disclosure may be implemented to alleviate one or more of these issues. Having multiple sets of projection image data acquired substantially simultaneously from multiple different angles/directions increases the possibility that at least one of them is not perturbed by higher noise. This way, compared to conventional approaches, more accurate 2D and 3D position data may be generated.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated herein. Although the terms “first” and “second” are used to describe various elements, these elements should not be limited by these terms. These terms are used to distinguish one element from another. For example, a first element may be referred to as a second element, and vice versa. Independent of the grammatical term usage, individuals with male, female or other gender identities are included within the term.

### Radiation Therapy System

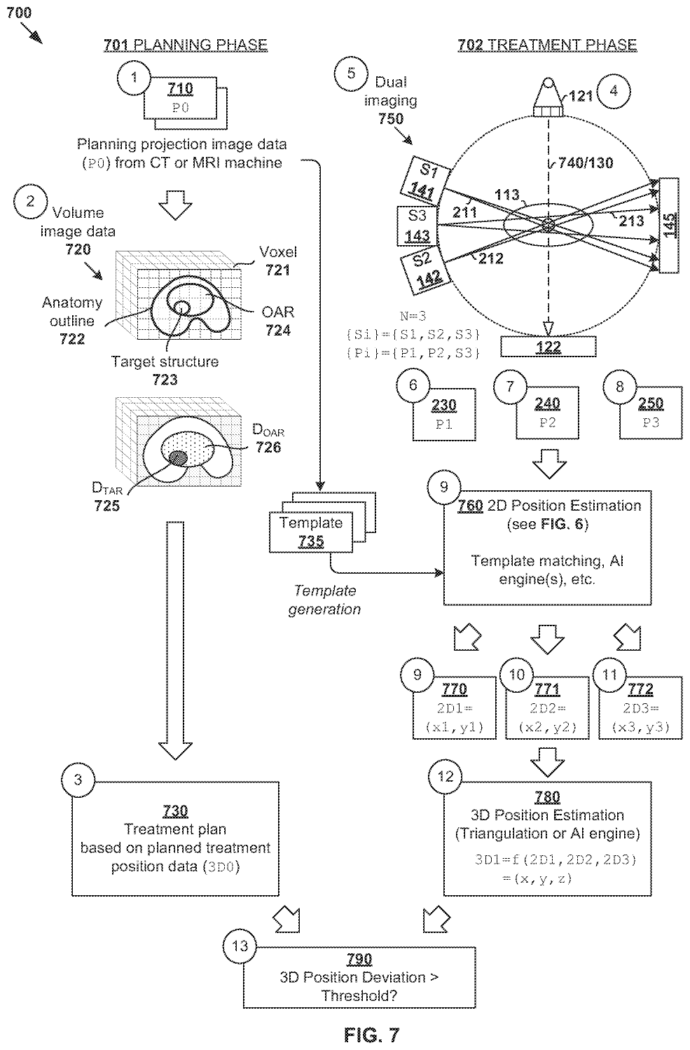

FIG. 1 is a schematic diagram illustrating example radiation therapy system 100 having a ring-based configuration to facilitate target structure tracking according to examples of the present disclosure. In this example, radiation therapy system 100 has a ring-based configuration that includes a circular gantry. In practice, any alternative configuration may be implemented, such as a C-arm configuration that includes a C-shaped gantry (to be described using FIG. 7). Radiation therapy system 100 may be configured to facilitate kilovolt (kV) imaging (see 140-145) during application of a megavolt (MV) treatment beam (see 130). Projection image data obtained using kV imaging may be subsequently used for target structure tracking according to examples of the present disclosure.

In the example in FIG. 1, radiation therapy system 100 may include gantry 110 having opening 111, patient support 112 (e.g., treatment couch) for supporting patient 113, control system 150 for controlling operation(s) of gantry 110 using drive system 115 and computer system 160 for, inter alia, target structure tracking according to examples of the present disclosure. During operation of radiation therapy system 100, gantry 110 may rotate about bore or opening 111 when actuated by drive system 115. To facilitate treatment delivery, radiation therapy system 100 may include a radiation source in the form of linear accelerator (LINAC) 121 as well as an imager/detector in the form of MV electronic portal imaging device (EPID) 122. LINAC 121 may generate and direct MV treatment beam 130 towards isocenter 114 through a target volume (i.e., patient 113) while gantry 110 rotates through a treatment arc. In practice, MV treatment beam 130 may be within a high-energy range, such as 1 mega-electron volts (MV) or greater.

Radiation therapy system 100 may further include kV imaging system 140 to facilitate target structure tracking during volumetric modulated arc therapy (VMAT), where gantry 110 rotates around patient 113 during radiation therapy. Imaging system 140 may include multiple kV imaging sources 141-143 and kV imager 145 (also known as an imaging panel or detector). Target structure tracking may also be performed during any other suitable treatment technique(s), such as static intensity modulated radiotherapy treatment (IMRT) that, for example, involves 5-12 fields at static gantry and is delivered with multileaf collimator (MLC), etc. In response to detecting imaging X-ray beams generated by imaging sources 141-143, imager 145 may generate suitable projection image data. Compared to LINAC 121, imaging sources 141-143 may be capable of producing imaging or diagnostic energy in the range of kV, such as below 160 kV. Imaging sources 141-143 may be configured to emit and direct respective imaging beams 211-213 towards different imaging areas of same imager 145. Example configurations of imaging system 140 will be discussed further below using FIGS. 2A-B.

Radiation therapy system 100 may be coupled with any suitable computer system(s) to facilitate treatment delivery and imaging, such as control system 150 and computer system 160 for target structure tracking. Control system 150 may be configured to generate and send control signal(s) to control the operations of LINAC 121 and imaging sources 141-143. Computer system 160 may be configured to obtain and process projection imaging data (see 171-172) from EPID 122 and imager 145. Projection image data 172 from imager 145 may be used to facilitate target structure tracking in 3D according to examples of the present disclosure. In practice, computer system 160 may be located in the same physical location as radiation therapy system 100 or connected to radiation therapy system 100 via any suitable communication network(s). Computer system 160 may be implemented using a physical machine (bare metal machine) and/or virtual machine that is deployed in a cloud-based environment. Control system 150 and computer system 160 may include any display device(s) and user input device(s), which are not shown for simplicity.

### kV Imaging System

Some example configurations of kV imaging system 140 in FIG. 1 will be described further using FIGS. 2A-B. FIG. 2A is a schematic diagram illustrating first example configuration 200 of imaging system 140 in FIG. 1. FIG. 2B is a schematic diagram illustrating second example configuration 203 of imaging system 140 in FIG. 1. In both examples, it should be noted that multiple imaging sources 141-143 are directed towards different imaging areas on the same imager 145 from different angles. In practice, the installation of multiple imagers may be relatively expensive. Further, there may be insufficient space to position an extra kV imager on radiation therapy system 100, which may have a ring-based configuration (as shown in FIGS. 2A-B) or C-arm configuration (see FIG. 7).

Depending on the desired implementation, imager 145 may be any suitable imaging panel that is capable of detecting imaging beams from multiple imaging sources 141-143 and generating projection image data based on the detections. Depending on how imaging sources 141-143 are mounted on radiation therapy system 100, imager 145 may have a substantially large area. Without limitations, one example may be the HyperSight™ Imager (available from Varian Medical Systems, Inc.) that is capable of producing high-definition images of a patient's anatomy and has a substantially large area (e.g., 86 cm×43 cm) to facilitate double imaging. Dual energy imaging techniques may also be implemented.

In a first example in FIG. 2A, third imaging source (denoted as “S3”) 143 may be a main imaging source (e.g., CBCT X-ray tube) on radiation therapy system 100, which is modified to include two extra kV imaging sources (i.e., X-ray tubes) capable of functioning as first and second imaging sources 141-142 (denoted as “S1” and “S2,” respectively). Any suitable separation angle between S1 141 and S3 143 (as well as S2 142 and S3 143) may be configured, such as a separation of minimum of 30 degrees (see 201-202) for the HyperSight™ Imager, etc. During patient setup, the main imaging source (e.g., CBCT X-ray tube) may be used to generate higher quality projection image data, such as to facilitate image registration prior to treatment delivery, etc.

S1 141 and S2 142 may each be positioned on one lateral side of S3 143. S1 141 may be configured to emit and direct a first imaging beam (see 211) towards a first imaging area (see 221) on one side of imager 145, thereby generating first projection image data (denoted as “P1” 230). S2 142 may be configured to emit and direct a second imaging beam (see 212) towards a second imaging area (see 222) on same imager 145, thereby generating second projection image data (denoted as “P2” 240). This way, S1 141 and S2 142 may image the same volume from different angles. During patient setup and/or subsequent target tracking, S3 143 may be configured to generate and direct a third imaging beam (see 213) towards a third imaging area (see 223) between first imaging area 221 and second imaging area 222, thereby generating third projection image data (denoted as “P3” 250). In practice, the quality of P3 250 may be higher than that of P1 230 and P2 240, such as to facilitate image registration prior to treatment delivery, etc.

In a second example in FIG. 2B, S1 141 may be an existing main imaging source (e.g., CBCT X-ray tube) on radiation therapy system 100, which is modified to include an additional X-ray tube that is capable of functioning as S2 142. Depending on the desired implementation, S1 141 and S2 142 in FIG. 2B may have a minimum separation angle of 15 degrees (see 204), etc. Similarly, S1 141 and S2 142 may be configured to image the same volume from different angles. For example, S1 141 may be configured to direct a first imaging beam (see 211) towards a first imaging area (see 261) on imager 145 to generate P1 230. S2 142 may be configured to direct a second imaging beam (see 212) towards a second imaging area (see 262) of same imager 145 to generate P2 240.

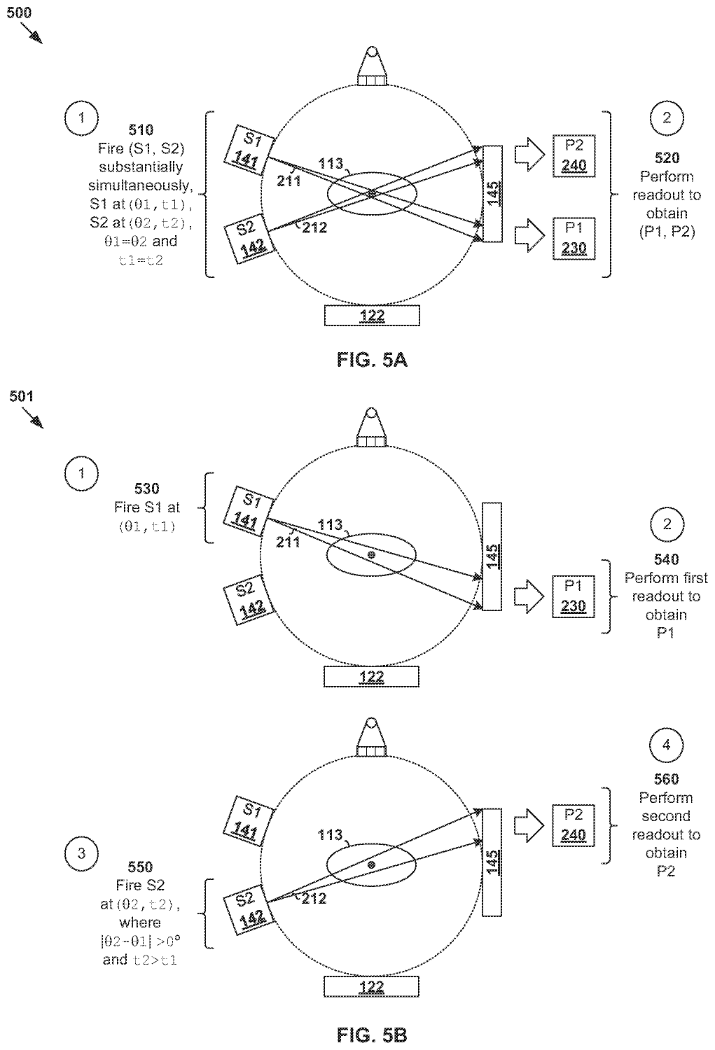

In both examples in FIGS. 2A-B, radiation therapy system 100 may include a first collimator associated with S1 141 and a second collimator associated with S2 142. Each collimator (not shown for simplicity) may be configured to change a characteristic (e.g., shape) of imaging beam 211/212. In practice, it is not necessary for S1 and S2 141-142 to image the entire diameter of patient 113. In one example (to be described using FIG. 5A), S1 and S2 141-142 (or all three sources 141-143) may be fired substantially simultaneously. In this case, S1 and S2 141-142 may be collimated to image only a small part or field of the patient's anatomy around isocenter 114, such as a field size of 7 cm×7 cm, 10 cm×10 cm, etc. In an alternative example (to be described using FIG. 5B), S1 and S2 141-142 (or all three sources 141-143) may be fired sequentially. In this case, S1 and S2 141-142 may be collimated to each have a larger field size, possibly partially overlapping each other depending on the desired implementation.

### Target Structure Tracking

According to examples of the present disclosure, target structure tracking may be performed in an improved manner based on multi-imaging of the same target volume from different angles using multiple imaging sources 141-142 (or 141-143) and imager 145. Examples of the present disclosure may be implemented to improve the accuracy of target structure tracking to reduce the probability of target miss and/or the probability of healthy tissue damage during treatment delivery. Examples of the present disclosure may be implemented as part of any suitable software suite for radiation therapy, such as the RapidTrack Realtime (RTR) suite from Varian Medical Systems, Inc. etc. RTR is software for continuous 3D position determination from fluoroscopic images.

In more detail, FIG. 3 is a schematic diagram illustrating example process 300 for computer system 160 to perform target structure tracking for radiation therapy. Example process 300 may include one or more operations, functions, or actions illustrated by one or more blocks, such as 310 to 341. Depending on the desired implementation, various blocks may be combined into fewer blocks, divided into additional blocks, and/or eliminated. Using the example in FIG. 1, blocks 310-341 may be performed using computer system 160 capable of functioning as a target structure tracking system. Computer system 160 may include any suitable module(s) or component(s) such as interface 161 to interface with radiation therapy system 100 to perform block 310, data processor 162 to perform blocks 320-340, etc.

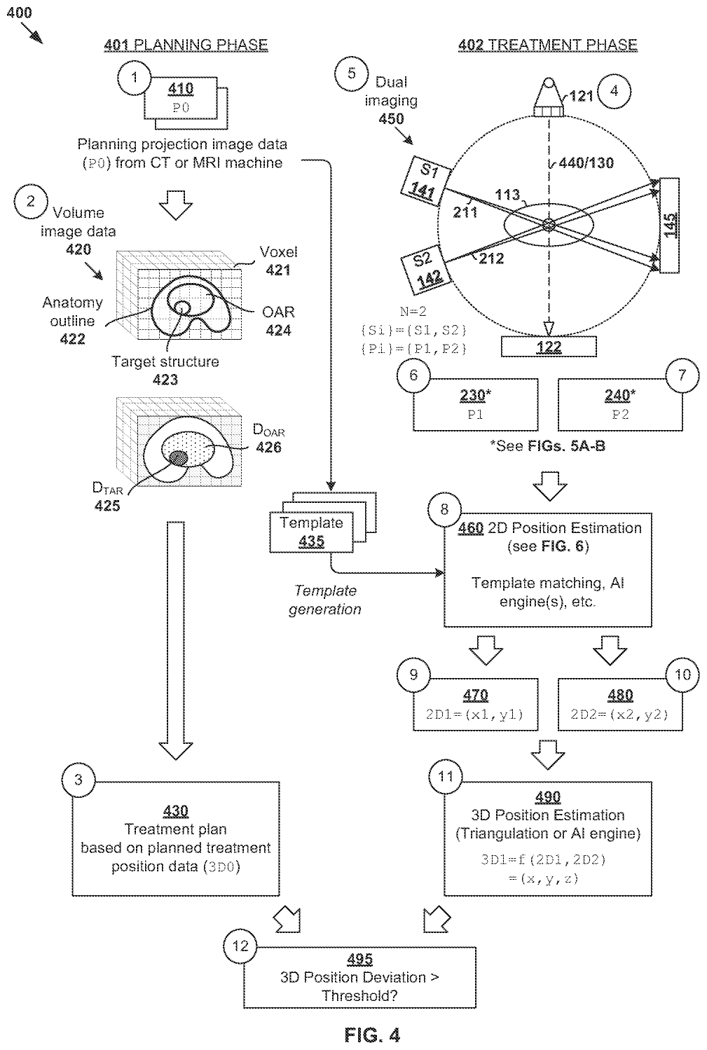

At 310 in FIG. 3, computer system 160 may obtain multiple (N) sets of projection image data denoted as {Pi}, where i=1, . . . , N and N≥2. In one example where N=2 (see 311 and FIG. 4), computer system 160 may obtain first projection image data=P1 230 and second projection image data=P2 240. In a second example where N=3 (see 312 and FIG. 7), computer system 160 may obtain P1 230, P2 240 and third projection image data=P3 250. Here, the term “obtaining” may refer generally to receiving or retrieving projection image data 230/240/250 from radiation therapy system 100, or any suitable data store or system in which projection image data 230/240/250 is stored. The term “projection image data” (used interchangeably with “2D projection data” and “2D projection image”) may refer generally to data representing properties of illuminating radiation rays transmitted through a subject (e.g., patient 113).

Each set of projection image data (Pi) may be generated using an imaging source (Si) and imager 145. In the examples in FIGS. 2A-B, P1 230 may be generated using first imaging source=S1 141 to emit first imaging beam 211 towards first imaging area 221/261 on imager 145 to image a target structure within patient 113 from a first angle (see 201/204). P2 240 may be generated using second imaging source=S2 142 to emit second imaging beam 212 towards second imaging area 222/262 on imager 145 to image the target structure from a second angle (see 202/204). Similarly, P3 250 may be generated using third imaging source=S3 143 to emit third imaging beam 213 towards third imaging area 223 on imager 145 to image the target structure from a third angle relative to first and second angles. Any suitable first angle and second angle may be configured. For example in FIG. 2A, first angle 201 and second angle 202 may be greater than 30 degrees from S3 143. In FIG. 2B, there may be a separation of more than 15 degrees between S1 141 and S2 142.

The multiple sets of projection image data {Pi} may be generated by firing associated imaging sources {Si} substantially simultaneously or sequentially (see 313). As will be described further using FIG. 5A for N=2, P1 230 and P2 240 may be generated by causing S1 141 to emit first imaging beam 211 and S2 142 to emit second imaging beam 212 substantially simultaneously; see also 311, 321 in FIG. 3. Alternatively, in FIG. 5B, P1 230 and P2 240 may be generated by sequentially causing S1 141 to emit first imaging beam 211 at a first time point (t1) and S2 142 to emit second imaging beam 212 at a second time point (t2>t1); see also 312, 322 in FIG. 3 and FIG. 5B. Here, the sequential firing of S1 141 and S2 142 may be configured such that motion (if any) of the target structure is substantially low or insignificant between the first time point (t1) and the second time point (t2) to improve the accuracy of subsequent 3D position data estimation.

At 320 in FIG. 3, computer system 160 may process projection image data {Pi}, including (a) P1 230 to determine first 2D position data associated with the target structure, and (b) P2 240 to determine a second 2D position data associated with the target structure. The first 2D position data associated with the target structure may be denoted as 2D1=(x1, y1), where x1 and y1 are respective x and y coordinates that are estimated based P1 230. The second 2D position may be denoted as 2D2=(x2, y2), where x2 and y2 are respective x and y coordinates that are estimated based on P2 240.

At 330 in FIG. 3, computer system 160 may estimate 3D position data (denoted as 3D1) associated with the target structure based on at least (2D1, 2D2), thereby tracking the target structure in 3D. Depending on the desired implementation, block 330 may involve performing triangulation (see 331) using any suitable algorithm(s) for estimating the 3D position, where 3D1=f ({2Di})=(x, y, z) in the lateral (x), longitudinal (y) and vertical (z) directions. Any suitable algorithm(s) or procedure(s) may be used to implement triangulation function (f), which will be discussed using FIG. 4. Alternatively, block 330 may involve estimating 3D position data by processing the 2D position data {2Di} using an AI engine that is trained to map input={2Di} to output=3D1 (see 332).

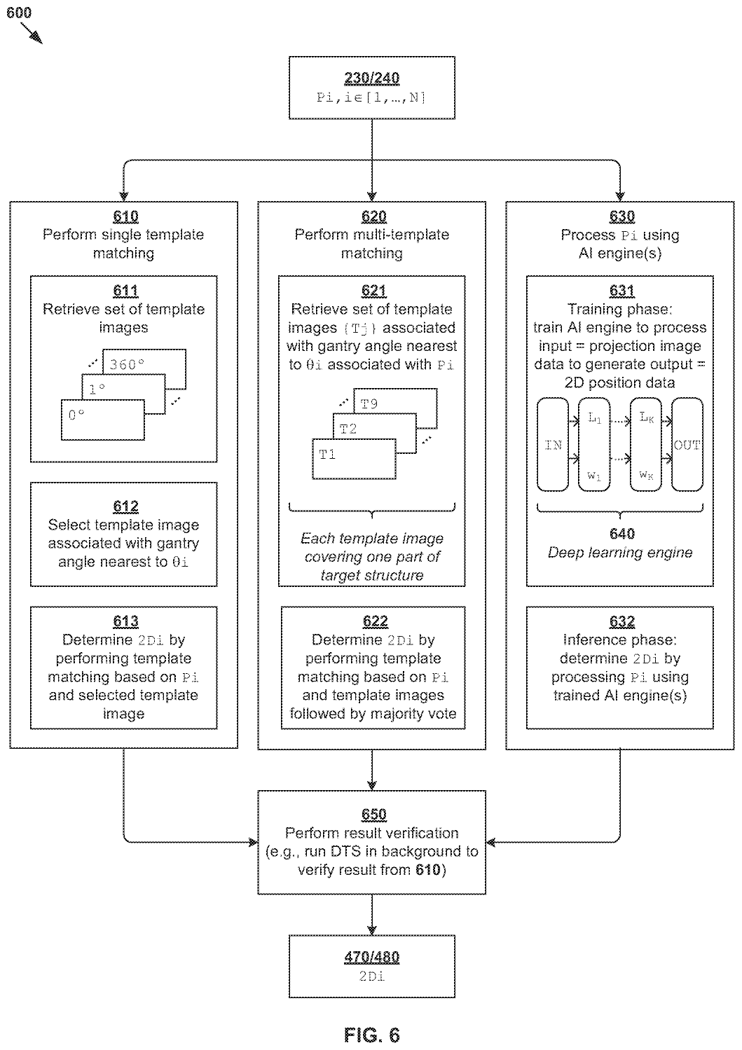

Some example approaches for block 320 will be described using FIG. 6. In one example (see 321 in FIGS. 3 and 610-620 in FIG. 6), computer system 160 may perform template matching to (a) match P1 230 to one or multiple first template images for a specific imaging angle to determine 2D1 and (b) match P2 240 to one or multiple second template images for the specific imaging angle to determine 2D2. Each of the multiple first/second template images may be associated with at least one part of the target structure. In another example (see 322 in FIGS. 3 and 630 in FIG. 6), computer system 160 may process P1 230 and P2 240 using an artificial intelligence (AI) engine that is trained to perform 2D position estimation to determine 2D1 and 2D2, thereby mapping input=Pi to output=2Di.

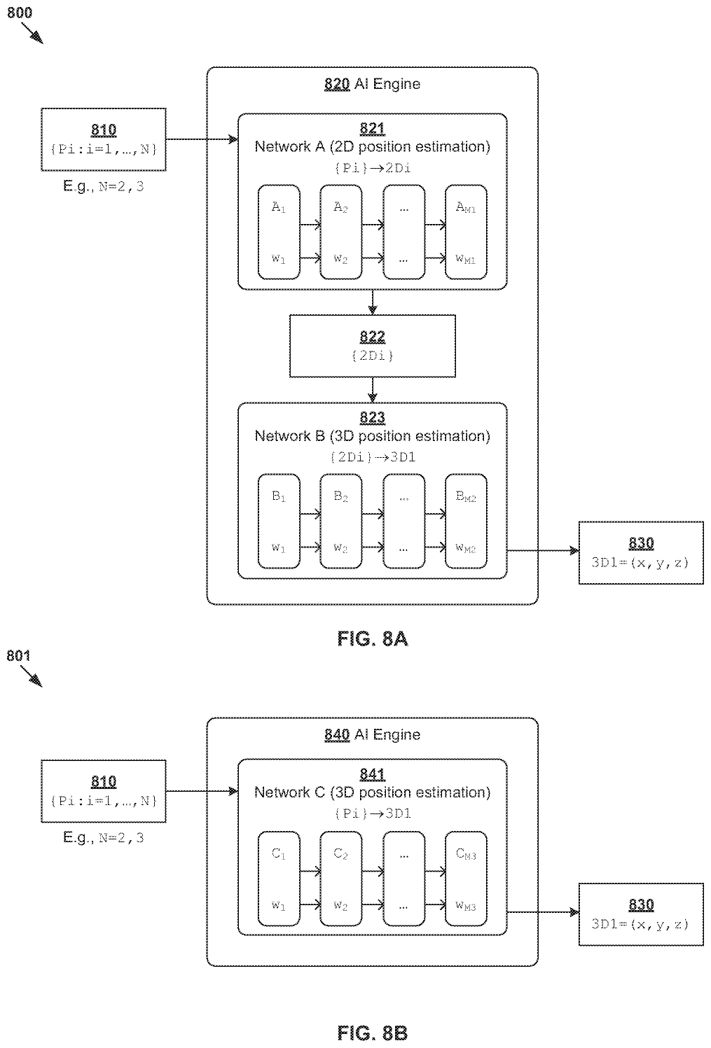

Alternatively, block 340 may be performed instead of template matching and triangulation according to blocks 320-330. In this case, at 340 in FIG. 3, computer system 160 may process multiple sets of projection image data {Pi} to determine 3D position data 3D1=(x, y, z) associated with the target structure. For example, {Pi} may be processed using an AI engine that is trained to perform 3D position estimation to map input={Pi} to output=3D1. Some examples will be explained using FIGS. 8A-B.

Examples of the present disclosure may be implemented as part of tumor position monitoring using either marker-based or markerless techniques. In the former case (i.e., marker-based), the “target structure” to be tracked may be a localizing fiducial marker that has been implanted into the patient's anatomy (e.g., near tumor), such as a radio-opaque marker, radiofrequency (RF) beacon, etc. In the latter case (i.e., markerless), the “target structure” may be a tumor, OAR, or any other structure (bony structure) of interest. In practice, multiple target structures may be imaged and tracked according to examples of the present disclosure. Various examples will be discussed below using FIGS. 4-9 with reference to an example treatment planning process.

### Example Treatment Planning Phase

Some examples relating to the acquisition and processing of N=2 sets of projection image data (i.e., P1 230 and P2 240) will be explained using FIG. 4. Here, FIG. 4 is a flowchart of example detailed process 400 for treatment planning and delivery. Example process 400 may include one or more operations, functions, or actions illustrated by one or more blocks, such as 410 to 495. Depending on the desired implementation, various blocks may be combined into fewer blocks, divided into additional blocks, and/or eliminated. In the example in FIG. 4, radiotherapy treatment may include a treatment planning phase (see 401) that involves generating a suitable treatment plan for patient 113 and a treatment delivery phase (see 402) that involves delivering treatment according to the treatment plan.

Examples of the present disclosure may be implemented during any suitable radiation therapy, such as stereotactic body radiation therapy (SBRT) for lung cancer treatment, etc. SBRT is a type of radiation therapy that delivers a high dose of radiation to a relatively small area of the body. For example, tumor tracking during lung SBRT may help to verify patient positioning during treatment to reduce the probability of a geographic miss by confirming that a tumor remains inside a planning target volume (PTV). In the following, an example target structure will be discussed with reference to a tumor. It should be noted that any other target structure(s) may be tracked.

At 410 in FIG. 1, image data acquisition may be performed to capture planning projection image data 410 (denoted as “P0”) associated with patient 113 (particularly the patient's anatomy). Any suitable medical image modality or modalities may be used, such as computed tomography (CT), magnetic resonance imaging (MRI), cone beam computed tomography (CBCT), positron emission tomography (PET), magnetic resonance tomography (MRT), single photon emission computed tomography (SPECT), any combination thereof, etc. For example, when CT or MRI is used, P0 410 may include a series of 2D images or slices, each representing a cross-sectional view of the patient's anatomy; volumetric or 3D images of the patient; or a time series of 2D or 3D images of the patient (e.g., four-dimensional (4D) CT or 4D CBCT). In practice, P0 410 may include transverse, coronal, and sagittal slices of the patient's anatomy.

At 420 in FIG. 4, treatment planning may be performed to, inter alia, generate a treatment plan that delivers a certain high dose to a target structure while delivering a lower dose to the OAR. . . . For example, segmentation (e.g., automated or manual) may be performed to generate volume image data 420 identifying various segments or structures based on P0 410. Volume image data 420 (also known as a digital or treatment volume) may be divided into multiple smaller volume-pixels (voxels) 421. Each voxel 421 may represent a 3D element within the treatment volume. Volume image data 420 may also include any suitable data relating to the contour, shape, size, and location of patient's anatomy 422, target structure 423 (e.g., tumor), OAR 424, or any other structure of interest (e.g., tissue, bone).

Further, dose calculation may be performed based on P0 410 and/or volume image data 420 to generate dose data specifying radiation doses to be delivered to target structure 423 (denoted “DTAR” at 425) and OAR 424 (denoted “DOAR” at 426). For example, target structure 423 may represent a malignant tumor requiring radiotherapy treatment, such as lung tumor, prostate tumor, etc. OAR 424 may be a proximal healthy structure that might be adversely affected by the treatment, such as central airway, rectum, bladder, etc. Target structure 423 is also known as a PTV. In practice, treatment volume 420 may include multiple targets 423 and OARs 424 with complex shapes and sizes. Although shown as having a regular shape (e.g., cube), voxel 421 may have any suitable shape (e.g., non-regular). Any additional and/or alternative data may be used, such as prescription data, disease staging data, biologic or radiomic data, genetic data, assay data, biopsy data, past treatment or medical history, any combination thereof, etc.

At 430 in FIG. 4, a treatment plan may be generated, such as based on a planned treatment position in 3D (denoted as 3D0) associated with target structure 423 that is estimated from P0 410 and volume image data 420. Depending on the desired implementation, treatment plan 430 may be generated to include 2D fluence map data for a set of beam orientations or angles. Each fluence map may specify the intensity and shape (e.g., as determined by an MLC) of a radiation beam emitted from a radiation source at a particular beam orientation and at a particular time. For example, IMRT or any other treatment technique(s) may involve varying the shape and intensity of the radiation beam while at a constant gantry and couch angle. Alternatively or additionally, treatment plan 430 may include machine control point data (e.g., jaw and leaf positions), VMAT trajectory data for controlling a treatment delivery system, etc. In practice, treatment plan 430 may be generated based on goal doses prescribed by a clinician (e.g., oncologist, dosimetrist, planner, etc.), such as based on the clinician's experience, the type and extent of the tumor, patient geometry and condition, etc. As will be described further below, template(s) 435 may be generated from volume image data 420 used for treatment planning, the contours of the target, isocenter of treatment plan 430, etc.

### Example Treatment Delivery Phase with Dual Imaging (N=2)

During treatment delivery phase 402, treatment plan 430 for patient 113 may be provided to radiation therapy system 100 in FIG. 1. Based on treatment plan 430, control system 150 in FIG. 1 may provide instruction(s) or control signal(s) 151 to, for example, to position LINAC 121 to apply a radiation dose to target structure 423 (i.e., tumor) while minimizing radiation dose to OAR 424, etc. Before treatment begins, patient 113 may be positioned in a supine position on treatment couch 112. To verify that patient 113 is in the correct position for treatment, image registration may be performed during patient setup.

In one example, a main CBCT radiation source (e.g., S3 143 in FIG. 2A) and imager 145 may be used to capture treatment projection image data such that the patient's current position on treatment couch 112 may be compared or registered against their planned treatment position. Where necessary, patient 113 may be repositioned to ensure that treatment is delivered to the intended target. During treatment delivery, gantry 110 may be rotated around patient 113 to deliver therapeutic radiation dose 440/130 to target structure 423 at various beam orientations according to treatment plan 430. During MV delivery, kV fluoroscopy with any fluoroscopic frequency (e.g., 0.5, 7 or 10 frames per second depending on the application and treatment speed) for tracking of target structure 423 may be implemented according to blocks 450-490 to reduce the risk of target miss and potentially improve treatment outcomes.

### (a) Dual Imaging

At 450 in FIG. 4, dual or double imaging on imager 145 may be initiated by control system 150 by generating and sending control signal(s) to radiation therapy system 100 to cause S1 141 and S2 142 to emit respective imaging beams 211-212 towards imager 145. In a first example, FIG. 5A is a schematic diagram illustrating example 500 for firing first and second imaging sources 141-142 substantially simultaneously. Here, S1 141 may be fired to emit first imaging beam 211 at θ1=first gantry angle and t1=first time point, and S2 142 fired to emit second imaging beam 212 at θ2=second gantry angle and t2=second time point. Using Aθ=|θ2−θ1| to denote the angular difference and Δt=t2−t1 to denote the time difference, Aθ˜0 and Δt˜0 may be implemented for the simultaneous approach. In practice, At may be as small as the time needed to read out imager 145. Computer system 160 may then perform a readout of imager 145 to obtain P1 230 and P2 240. See 510-520 in FIG. 5A.

According to a second example, FIG. 5B is a is a schematic diagram illustrating example 501 for firing first and second imaging sources 141-142 sequentially. First, control system 150 may be configured to cause S1 141 to emit first imaging beam 211 at (θ1, t1), followed by computer system 160 performing a first readout to obtain P1 230. Next, control system 150 may be configured to cause S2 142 to emit second imaging beam 212 at (θ2, t2), followed by computer system 160 performing a second readout to obtain P2 240. As such, Δθ=|θ2−θ1|>0 and Δt=t2−t1 >0 for the sequential approach. See 530-560 in FIG. 5B.

The examples in FIGS. 5A-B should be contrasted against conventional approaches that rely on a pair of kV imaging source and kV detector/imager to acquire kV fluoroscopic images at different angles in time. In this case, template matching (if goes well) results in a 2D position and requires triangulation with earlier data (i.e.,≥20 degrees earlier) to determine a 3D position. As discussed, triangulation with earlier data may result in the wrong 3D position if the target structure has moved in the time between the two images. To remove the time element that reduces the accuracy of triangulation, the simultaneous approach in FIG. 5A may be implemented to generate P1 230 and P2 240 at substantially the same time. In this case, there should be sufficient distance on imager 145 between P1 230 and P2 240 in order not to contribute undesirable amount of scatter to each other. For example, S1 141 and S2 142 may be configured to image a small field (e.g., 7 cm×7 cm) of the patient's anatomy around isocenter 114.

Alternatively, the example in FIG. 5B may be implemented to generate P1 230 and P4 240 sequentially. Here, At may be configured to be “sufficiently low” such that the probability of motion of target structure 423 affecting the tracking accuracy is reduced compared to the conventional approaches that necessitate ≥20° separation in gantry angles. For example, time difference Δt=t2−t1 may be around few tens of milliseconds (ms). For a gantry speed of 30° per second and Δt=20 ms, the angular difference Ae may be around 0.6°, which is significantly less than ≥20° required in conventional approaches. In other words, Δt may be configured such that any motion of target structure 423 during At would be substantially insignificant and have little or no impact on the accuracy of subsequent triangulation. The sequential approach in FIG. 5B may be implemented such that P1 230 and P4 240 will contribute little or no scatter to each other. In this case, P1 230 and P4 240 may be closer together on imager 145, which takes away some limitations of where S1 141 and S2 142 may be positioned.

### (b) 2D Position Data Estimation

At 460, 470 and 480 in FIG. 4, computer system 160 may process P1 230 and P2 240 to (a) determine a first 2D position denoted as 2D1=(x1, y1) associated with target structure 423 based on P1 230 and (b) determine a second 2D position denoted as 2D2=(x2, y2) associated with target structure 423 based on P2 240. Here, (2D1, 2D2) are associated with a specific gantry angle at which respective P1 230 and P2 240 are acquired from different directions/angles shown in FIGS. 2A-B and FIGS. 5A-B. In practice, each 2D position may represent the 2D position of a representative point of target structure 423, such as the centroid position, etc. In practice, any suitable 2D position estimation approach may be used, some examples of which will be described using FIG. 6 below.

### Example 2D Position Data Estimation

FIG. 6 is a schematic diagram illustrating example 2D position estimation 600 to facilitate target structure tracking in 3D. Example approaches to be discussed below include single template matching (see 610), multi-template matching (see 620), and AI approach (see 630). For simplicity, the examples will be explained below with reference to Pi=i^{th} set of projection image data that is generated using imaging source=Si 14i and imager 145 and associated with gantry angle θi, where i∈[1, . . . , N] and N≥2. Pi=projection image data may also be referred to as an online image that is obtained between the delivery of treatment radiation beams. Note that the following approaches may be implemented for any suitable N≥2. Some examples will be explained below using N=2 to determine (a) first 2D position data (2D1) based on P1 230 and (b) second 2D position data (2D2) based on P2 240. Depending on the desired implementation, one approach may be selected as the main approach, while at least one other approach may be implemented for verification purposes.

### (a) Single Template Matching

At 610 in FIG. 6, a first approach may involve single template matching. At 611, computer system 160 may retrieve a set of template images (see 435 in FIG. 4) that are generated based on planning projection image data (P0) 410 in FIG. 4. Any additional information may also be used to generate set 435, such as information relating to contoured structures (e.g., drawn by physician or determined using software/AI engine(s) for segmentation), isocenter of treatment plan 430, etc. In one example (shown in FIG. 6), in order to be able to track from all gantry angles, a set of K template images may be generated for K=360 gantry angles that are spaced at L=1 degree.

Next, at 612 in FIG. 6, computer system 160 may select, from set 435, a particular template image associated with a gantry angle that is nearest to or best matches ei. Further, at 613, computer system 160 may perform template matching by matching Pi matched against the selected template image to identify the target structure. In practice, Pi may be pre-processed (e.g., filtered) prior to template matching to enhance the visibility of the target structure in Pi. Template matching may involve calculating a normalized cross correlation within a specific search region around the isocenter, which results in a match score value between 0 and 1. Calculating the match score at different pixel offsets produces a match score surface defined over the search region. A possible match may be indicated by the highest peak in the match score surface. However, in practice, this may be an incorrect match (especially for noisy images with little contrast). For each match, a peak-to-sidelobe-ratio (PSR) may also be calculated, where the PSR is the peak value divided by the standard deviation of the sidelobes. A minimum threshold for the match score value and PSR may be used to reject likely false matches. The 2D position data (2Di) associated with target structure 423 may then be determined based on the match.

### (b) Multi-Template Matching

At 620 in FIG. 6, a second approach for 2D position estimation may involve template matching based on multiple template images that each cover one part or portion of target structure. Here, multiple templates may be used to reduce the risk of having one template match resulting in a wrong position. For example, computer system 160 may perform multi-template matching to (a) match P1 230 to multiple first template images for a specific imaging angle to determine multiple positions/instances of 2D1 and (b) match P2 240 to multiple second template images to determine multiple positions/instances of 2D2. This may be followed by a majority vote over the multiple positions/instances of 2D1 and over the multiple positions/instances of 2D2.

For example, P1 230 may be matched against N=9 template images and P2 240 against M=9 template images (e.g., labelled “T1” to “T9”). A majority vote on the ensemble of tracking results may be performed to reduce or avoid incidental outliers of template matching, thereby reducing the risk of an incorrect match. For example, in noisy images, one template match could incidentally lead to a wrong position, and the majority vote over multiple matches may improve the matching result. In practice, template matching according to blocks 610-620 may have a number of limitations, such as when the tumor is small, the tumor has limited contrast with surrounding tissue(s), the tumor is at least partly obscured by over projecting structures (e.g., spine, ribs), the kV images are relatively noisy due to insufficient x-ray transmission etc. In some scenarios, an AI-based approach according to block 630 may be used to potentially improve tracking results.

### (c) AI-Based Approach

At 630 in FIG. 6, a third approach for 2D position estimation from {Pi}=multiple sets of projection image data (e.g., kV images) may involve AI engine(s). As used herein, the term “AI engine” may refer to any suitable hardware and/or software component(s) of a computer system that are capable of executing algorithms according to any suitable AI model(s). Depending on the desired implementation, “AI engine” may be a machine learning engine based on machine learning model(s), deep learning engine based on deep learning model(s), etc. In general, deep learning is a subset of machine learning in which multi-layered neural networks may be used for feature extraction as well as pattern analysis and/or classification.

Depending on the desired implementation, any suitable AI model(s) may be used, such as convolutional neural network, recurrent neural network, deep belief network, generative adversarial network (GAN), autoencoder(s), variational autoencoder(s), long short-term memory architecture for tracking purposes, or any combination thereof, etc. In practice, a neural network is generally formed using a network of processing elements (called “neurons,” “nodes,” etc.) that are interconnected via connections (called “synapses,” “weight data,” etc.). A processing layer of a convolutional neural network may be a convolutional layer, pooling layer, un-pooling layer, rectified linear units (ReLU) layer, fully connected layer, loss layer, activation layer, dropout layer, transpose convolutional layer, concatenation layer, or any combination thereof, etc. For example, convolutional neural networks may be implemented using any suitable architecture(s), such as UNet, LeNet, AlexNet, ResNet, VNet, DenseNet, OctNet, etc.

In the example in FIG. 6, a deep learning engine (see 640) that includes a hierarchy of multiple (K) processing layers (denoted as (L₁ to L_{K}) may be used, such as an input layer, an output layer, and multiple (i.e., two or more) “hidden” layers between the input and output layers. The processing layers (L₁ to L_{K}) are associated with respective weight data (w1 to wK). In one example, processing layers may be trained from end-to-end (e.g., from the input layer to the output layer) to extract feature(s) from an input and classify the feature(s) to produce an output. In particular, during a training phase at 631 in FIG. 6, deep learning engine 640 may be trained to process input=projection image data (Pi) that is acquired using imaging system 140 to generate output=2D position data (2Di) associated with a target structure. This way, during an inference phase at 632, computer system 160 may process input=Pi using trained deep learning engine 640 to estimate output=2Di.

Deep learning engine 640 may be trained prior to treatment delivery phase 402 using any suitable approach, such as supervised learning, unsupervised learning, semi-supervised learning, reinforcement learning, etc. For example, in supervised learning, deep learning engine 640 may be trained on a dataset of labeled examples in order to learn the relationship between input=Pi showing at least part of a target structure and output=2Di associated with the target structure. Any suitable training data may be used, such as synthetic data such as digitally reconstructed radiographs (DRRs), real patient data, or a combination of both. Deep learning engine 640 may be trained using training data that is specific to patient 113, or a large variation of possible patients. In practice, a patient-specific training strategy may tackle the issue of inter-patient and inter-tumor variations (e.g., tumor size, shape, location, motion). In this case, DRRs may be synthetically generated for every degree of a full gantry arc (360°) based on planning projection image data (P0) 410.

Alternatively, in unsupervised learning, deep learning engine 640 may be trained on a dataset of unlabeled examples in order to learn patterns and relationships in the data without any prior knowledge of the output labels. In semi-supervised learning, both labeled and unlabeled data may be used. Semi-supervised learning is useful in situations where there is a large amount of unlabeled data available, but it might be too expensive or difficult to label all of it. In reinforcement learning, deep learning engine 640 may learn to perform 2D position data estimation by trial and error where it is rewarded for taking actions that lead to desired outcomes and penalized for taking actions that lead to undesired outcomes.

Without limitations, deep learning engine 640 may be a deep Siamese neural network that includes two identical sub-networks that are trained on the same dataset. The twin subnetworks may share weights among themselves and the loss is computed using the information from two different images to learn a similarity measure between objects. Various implementation details of a deep Siamese neural network for tumor tracking may be found in the following article, which is incorporated herein by reference: Grama D, Dahele M, van Rooij W, Slotman B, Gupta D K, Verbakel W F A R, “Deep learning-based markerless lung tumor tracking in stereotactic radiotherapy using Siamese networks,” Med Phys, 2023 Nov; 50(11): 6881-6893.

### (d) Result Verification

At 650 in FIG. 6, computer system 160 may perform result verification using any suitable approach. For example, one of approaches 610-630 (e.g., single template matching) may be selected as a main approach, while at least one other is used to verify the result of the main approach. In the case of single template matching 610, computer system 160 may additionally run a digital tomosynthesis (DTS) process in the background as verification, etc. The result of the DTS process may be used in response to determination that the result from single kV tracking is insufficient. In practice, DTS may combine multiple previous images to reduce noise and contrast in one plane. In response to determination that the result from single kV tracking is insufficient, becomes uncertain or deviates too much from the DTS result, the DTS result may be presented instead. By using multiple previous images, DTS may increase the visibility of the target while blurring over-projecting structures to improve the accuracy of tumor monitoring. Any additional and/or alternative approach for result verification may be performed.

### Example 3D Position Data Estimation

Referring to FIG. 4 again, at 490, based on {2Di}=(2D1, 2D2) associated with the target structure, computer system 160 may determine 3D position data denoted as 3D1=(x, y, z), such as a 3D centroid position associated with target structure 423. In practice, the 2D matches may determine the 2D centroid position (e.g., tumor centroid position) for the respective imaged planes, and triangulation or an AI engine may be used to determine the third dimension.

### (a) Triangulation (Related to 331 in FIG. 3)

In one approach, computer system 160 may implement any suitable triangulation technique(s) to determine 3D1 based on {2Di}. The specific formula(s) for triangulation may depend on the geometry data relating to the spatial location of S1 141 and S2 142 relative to imager 145 or isocenter 114. For example, since the respective positions of S1 141 and S2 142 are fixed (known), the respective positions of P1 230 and P2 240 on imager 145 are also fixed (known). As such, for each projection image data 230/240, computer system 160 may determine the correct location of isocenter 114 on imager 145. After performing a readout of imager 145, computer system 160 may determine the 3D position data of target structure 423 relative to isocenter 114.

One example for triangulation may involve constructing a ray (Ri) from a particular imaging source Sᵢ to 2Di=centroid position for projection image data Pi. For the case of N=2 in FIG. 4, a first ray (R1) may be constructed from source=S1 141 to 2D1=(x1, y1) associated with P1 230 and a second ray (R2) from source=S2 142 to 2D2=(x2, y2) associated with P2 240. In practice, these rays/lines may not intersect. In this case, the point of nearest approach between R1 and R2 may be computed. In particular, the point with the median position along the direction of R1 associated with P1 230 may be selected as the 3D position of the centroid. Next, the 3D position may be projected onto a plane associated with P2 240. Defining 3D1=(x, y, z) and 2Di=(xi, yi), the centroid position on the image plane may be determined using the following:

xi=(x cos θ−z sin θ)*M and yi=y*M. (1)

In equation (1), x may point toward the right, y toward the gantry and z toward the ceiling. (xi, yi) are coordinates of the centroid on the image plane with x-ray central axis as origin where xi points toward the image right and yi toward the gantry. Note that M=(s+d)/(s−x*sin θ−z*cosθ), where s=source-to-isocenter distance, d=isocenter-to-detector distance and θ=angle of incidence associated with imaging source Si (e.g., measured from the 12 o'clock position in the clockwise direction when facing the gantry). In practice, triangulation between near simultaneously acquired projection image data {Pi} should provide a perfect third dimension. Even for lung tumors, N sets of projection image data may be acquired substantially simultaneously in the same breathing phase, and by definition, they reflect the same position of the target structure.

However, there is a chance that one of the matches is not accurate, such as when the projection image data is very noisy. For example, if the match is 2 mm off, triangulation with only one other set of projection image data could result in a much larger error for the third dimension. This may result in a much larger change in position with regard to the previous 3D position than would be expected from the imaging frequency. In this case, one approach to improve accuracy is to apply a sequential stereo technique in the background to, for each of the matches (2D1,2D2), perform a sequential stereo with previous matches. This may involve calculating an epipolar distance that is measured in a direction that is perpendicular to both (a) a first line that extends from S1 141 to 2D1=(x1, y1) associated with a target structure in P1 230 and (b) a second line that extends from S2 142 to 2D2=(x2, y2) associated with the same target structure in P2 240. The epipolar distance may be used to determine the 3D position data. It may also be used to reject a possible inaccurate match, which would result in a larger epipolar distance.

### (b) AI-Based Approach

In another approach, computer system 160 may implement or access an AI engine to determine 3D1 based on {2Di} and possibly additional data, such as known geometry data relating to the spatial location of S1 141 and S2 142 relative to imager 145 or isocenter 114. The AI engine may be trained to map input={2Di} to output =3D1 associated with target structure 423. Similar to example AI engine 640 in FIG. 6, the AI engine for 3D position data estimation may include multiple processing layers associated with a set of weight data. The AI engine may be trained using any suitable approach, such as supervised learning, semi-supervised learning, unsupervised learning, reinforcement learning, etc. Various implementation details explained using FIG. 6 are also applicable here and will not be repeated for brevity.

### (c) Online Adjustment(s)

At 495 in FIG. 4, computer system 160 may determine whether adjustment(s) are needed by comparing (a) the estimated 3D position data 3D1=(x, y, z) calculated at block 490 with the (b) planned treatment position data 3D0 based on which treatment plan 430 is generated. In response to determination that the difference/deviation between 3D1 and 3D0 exceeds a predetermined threshold, computer system 160 may generate an alert that an adjustment is required. A deviation that exceeds the predetermined threshold may indicate that a significant portion of target structure 423 is extending outside of a threshold region.

Based on the deviation detected, computer system 160 may determine or estimate adjustment(s) to the patient setup (e.g., position or orientation of couch 112) and/or treatment beam 440/130 (e.g., gantry angle, collimator setup) and/or instructions to the patient about the depth of breathing or depth of a breath hold in order to achieve the best match between treatment geometry (i.e., current 3D position data) and the planned geometry (i.e., planned treatment position data). Where applicable, treatment may be interrupted. Using examples of the present disclosure, positional verification may be performed during radiation treatment to identify patients who move more than a predetermined threshold, enabling adjustment(s) during treatment delivery phase 402 to achieve better treatment outcomes for patient 113.

### Example Treatment Phase with Triple Imaging (N=3)

Some examples relating to the acquisition and processing of N=3 sets of projection image data (i.e., P1 230, P2 240 and P3 250) will be explained using FIG. 7. In general, compared to the case of N=2 in FIG. 4, the accuracy of target structure tracking may be improved based on additional projection image data (i.e., P3 250). Here, FIG. 7 is a flowchart of second example detailed process 700 for treatment planning and delivery. Example process 700 may include one or more operations, functions, or actions illustrated by one or more blocks, such as 710 to 790. Depending on the desired implementation, various blocks may be combined into fewer blocks, divided into additional blocks, and/or eliminated.

In the example in FIG. 7, radiotherapy treatment may include a treatment planning phase (see 701) that involves generating a suitable treatment plan for patient 113 and a treatment delivery phase (see 702) that involves delivering treatment to target structure 723 according to the treatment plan. Blocks 710-730 in FIG. 7 are similar to corresponding blocks 410-430 in FIG. 4, the implementation details of which are applicable here and will not be repeated for brevity.

### (b) Triple kV Imaging

At 750 in FIG. 7, triple imaging on imager 145 may be initiated by control system 150 by causing S1 141, S2 142 and S3 143 to emit respective imaging beams 211-213 towards imager 145. Similar to the examples in FIGS. 5A-B, imaging sources 141-143 may be fired substantially simultaneously or sequentially. For example, S1 141 may be fired to emit first imaging beam 211 towards first imaging area 221 on imager 145 at (θ1=first gantry angle, t1=first time point). S2 142 may be fired to emit second imaging beam 212 towards second imaging area 222 on imager 145 at (θ2=second gantry angle, t2=second time point). S3 143 may be fired to emit third imaging beam 213 towards third imaging area 223 on imager 145 at (θ3=third gantry angle, t3=third time point).

The angular difference and time difference between the firing may be respectively denoted as Δθ=|θi−θj| and Δt=tj−ti, where i, j∈[1, 2, 3] and i≠j. For the simultaneous or near simultaneous approach, Δθ˜0 and Δt˜0 may be implemented. In practice, Δt may be as small as the time needed to read out imager 145. For the sequential approach, Δθ>0 and Δt>0 may configured such that any motion of target structure 723 during Δt would be substantially insignificant and have little or no impact on the accuracy of subsequent triangulation. Computer system 160 may then perform a readout of imager 145 to obtain P1 230, P2 240 and P3 250.

(b) 2D position Data Estimation

At 760 in FIG. 7, computer system 160 may perform 2D position data estimation by processing {Pi, i=1, 2, 3}, particularly P1 230, P2 240 and P3 250. This is to determine (a) 2D1=(x1, y1) representing first 2D position data associated with target structure 423 based on P1 230, (b) 2D2=(x2, y2) representing second 2D position data associated with target structure 423 based on P2 240, and (c) 2D3=(x3, y3) representing third 2D position data associated with target structure 423 based on P3 250. See 770-772 in FIG. 7.

2D position estimation may be performed using the examples in FIG. 6, such as single template matching (see 610), multi-template matching (see 620) and AI engine processing (see 630). Various implementation details described using FIG. 6 are applicable here and will not be repeated for brevity. Note that when three imaging sources 211-213 are used, result verification at block 650 may involve DTS using three sets of projection image data (i.e., P1 230, P2 240 and P3 250). In this case, it may not be necessary to use previous images for the DTS.

### (c) 3D Position Data Estimation

At 780 in FIG. 7, based on (2D1, 2D2, 2D3) associated with target structure 723, computer system 160 may determine 3D position data denoted as 3D1=(x, y, z), such as a centroid position associated with target structure 423. In practice, the 2D matches may determine this centroid position (e.g., tumor centroid position) for the respective imaged planes, and triangulation is used to determine the third dimension. In one approach, triangulation may be performed to determine 3D1 based on (2D1, 2D2, 2D3). The specific technique(s) for triangulation may depend on the geometry data relating to the spatial location of S1 141, S2 142 and S3 143 relative to imager 145 or isocenter 114. Alternatively, an AI engine may be trained to map input=(2D1, 2D2, 2D3) to output=3D1. Various details relating to block 490 in FIG. 4 are applicable here and will not be repeated for brevity.

At 790 in FIG. 7 computer system 160 may determine whether adjustment(s) are needed by comparing (a) the estimated 3D position data 3D1=(x, y, z) calculated at block 780 with the (b) planned treatment position data 3D0 based on which treatment plan 735 is generated. In response to determination that the difference/deviation between 3D1 and 3D0 exceeds a predetermined threshold, computer system 160 may generate an alert that an adjustment is required. Based on the deviation, computer system 160 may determine or estimate adjustment(s) to the patient setup and/or treatment beam 440/130. Where applicable, treatment may be interrupted. Using examples of the present disclosure, positional verification may be performed based on P1 230, P2 240 and P3 250 acquired during treatment delivery phase 402 to achieve better treatment outcomes for patient 113.

### AI engine(s) for 3D Position Data Estimation

3D position data estimation according to block 340 in FIG. 3 will be explained further using FIGS. 8A-8B. Using the examples in FIGS. 8A-B, multiple sets of projection image data {Pi} may be processed to determine 3D position data associated with a target structure, where i=1, . . . , N. In this case, it is not necessary to perform 2D position data estimation and triangulation to determine the 3D position data (see blocks 460-490 in FIG. 4 and blocks 760-780 in FIG. 7).

FIG. 8A is a schematic diagram illustrating first example 800 of 3D position data estimation using AI engine 820. Here, first AI engine 820 may be implemented to process input={Pi} and map it to output=3D position data denoted as 3D1. For example, {Pi} may include P1 230 and P2 240 for N=2, and additionally P3 250 for N=3. Depending on the desired implementation, AI engine 820 may be a deep learning engine includes a first network labelled “A” (see 821) to perform 2D position data estimation to determine 2Di=(xi, yi) associated with Pi (see 822). Network “A” 821 may include multiple (M1) processing layers denoted as A₁, A₂, . . . , A_{M1}. AI engine 820 may further include a second network labelled “B” (see 823) to perform 3D position data estimation to determine 3D1=(x, y, z) based on {2Di} for i=1, . . . , N (see 823). Network “B” 823 may include multiple (M2) processing layers denoted as B₁, B₂, . . . , B_{M2}.

During training, first AI engine 820 may learn first weight data (w₁, w₂ . . . , w_{M1}) associated with (A₁, A₂, . . . ,A_{M1}) to perform 2D position data estimation based on {Pi} and second weight data (w₁, w₂ . . . , w_{M2}) associated with (B₁, B₂, . . . , BM₂) to perform 3D position data estimation based on {2Di}. Network “A” 821 and network “B” 823 may be trained together, or separately. In practice, network “A” 821 may be deployed to perform 2D position data estimation (i.e., in place of template matching) according to block 630 in FIG. 6. Similarly, network “B” 823 may be deployed (i.e., separately from network “A” 821) to perform 3D position data estimation using the result of template matching and triangulation according to block 490 in FIG. 4 and block 780 in FIG. 7.

FIG. 8B is a schematic diagram illustrating second example 801 of 3D position data estimation using AI engine 840. Here, second AI engine 840 may be implemented to process and map input={Pi} to output=3D1. Similarly, {Pi} may include P1 230 and P2 240 for N=2, and additionally P3 250 for N=3. Compared to example in FIG. 8A, second AI engine 840 in FIG. 8B may include one network labelled “C” (see 841) to perform 3D position data estimation based on {Pi}. Network “C” 841 may include multiple (M3) processing layers denoted as C₁, C₂, . . . , C_{M3}. During training, AI engine 840 may learn weight data (w₁, w₂ . . . , w_{M3}) associated with (C₁, C₂, . . . , C_{M3}) to perform 3D position data estimation based on {Pi}.

Similar to deep learning engine 640 in FIG. 6, any suitable AI model(s) may be implemented for network 821/823/841, such as a deep Siamese neural network that includes two identical sub-networks that are trained on the same dataset. AI engine 820/840 may be trained using any suitable approach, such as supervised learning, semi-supervised learning, unsupervised learning, reinforcement learning, or any combination thereof. Various implementation details relating to AI engine 640 in FIG. 6 are also applicable to AI engine 820/840 and will not be repeated here for brevity.

### Example C-Arm Configuration

Although discussed using radiation therapy system 100 having a ring-based configuration, any alternative configuration may be used. For example, FIG. 9 is a schematic diagram illustrating example radiation therapy system 900 having a C-arm configuration to facilitate target structure tracking for radiation therapy. In this example, example radiation therapy system 900 may include C-arm gantry 910, therapeutic radiation source in the form of LINAC 921, EPID (not shown) and treatment couch 912 to support a patient (not shown). Similar to the example in FIG. 1, example radiation therapy system 900 may be configured with an imaging system that includes multiple imaging sources 141-143 and imager 145. The extra imaging sources 141-142 may require an extra frame attached to main imaging source 143.

Radiation therapy system 900 may further include control system 150 to generate and send control signal(s) for positioning and causing multiple imaging sources 141-143 to emit imaging beams towards imager 145 to image target structure(s) within a patient from different angles. Computer system 160 may be deployed to obtain P1 230 and P2 240 generated using multiple imaging sources 141-142 and to perform target structure tracking according to the examples discussed using FIG. 1 to FIG. 8B.

### Computer System

The above examples can be implemented by hardware (including hardware logic circuitry), software or firmware or a combination thereof. The above examples may be implemented by any suitable computing device, computer system, etc. The computer system may include processor(s), memory unit(s) and physical NIC(s) that may communicate with each other via a communication bus, etc. The computer system may include a non-transitory computer-readable medium having stored thereon instructions that, when executed by the processor, cause the processor to perform processes described herein with reference to the drawings.

The techniques introduced above can be implemented in special-purpose hardwired circuitry, in software and/or firmware in conjunction with programmable circuitry, or in a combination thereof. Special-purpose hardwired circuitry may be in the form of, for example, one or more application-specific integrated circuits (ASICs), programmable logic devices (PLDs), field-programmable gate arrays (FPGAs), and others. The term ‘processor’ is to be interpreted broadly to include a processing unit, ASIC, logic unit, or programmable gate array etc.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or any combination thereof.

Those skilled in the art will recognize that some aspects of the embodiments disclosed herein, in whole or in part, can be equivalently implemented in integrated circuits, as one or more computer programs running on one or more computers (e.g., as one or more programs running on one or more computing systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure.

Software to implement the techniques introduced here may be stored on a non-transitory computer-readable storage medium and may be executed by one or more general-purpose or special-purpose programmable microprocessors. A “computer-readable storage medium”, as the term is used herein, includes any mechanism that provides (i.e., stores and/or transmits) information in a form accessible by a machine (e.g., a computer, network device, personal digital assistant (PDA), mobile device, manufacturing tool, any device with a set of one or more processors, etc.). A computer-readable storage medium may include recordable/non recordable media (e.g., read-only memory (ROM), random access memory (RAM), magnetic disk or optical storage media, flash memory devices, etc.).

The drawings are only illustrations of an example, wherein the units or procedure shown in the drawings are not necessarily essential for implementing the present disclosure. Those skilled in the art will understand that the units in the device in the examples can be arranged in the device in the examples as described or can be alternatively located in one or more devices different from that in the examples. The units in the examples described can be combined into one module or further divided into a plurality of sub-units.

1. A method for a computer system to perform target structure tracking for radiation therapy, wherein the method comprises:
obtaining first projection image data that is generated using a first imaging source to emit a first imaging beam towards a first imaging area on an imager to image a target structure within a patient from a first angle;
obtaining second projection image data that is generated using a second imaging source of the radiation therapy system to emit a second imaging beam towards a second imaging area on the imager to image the target structure from a second angle;
processing (a) the first projection image data to determine first two-dimensional (2D) position data associated with the target structure and (b) the second projection image data to determine second 2D position data associated with the target structure; and
based on at least the first 2D position data and the second 2D position data, determining three-dimensional (3D) position data associated with the target structure, thereby tracking the target structure in 3D.

2. The method of claim 1, wherein obtaining the first projection image data and the second projection image data comprises:
obtaining the first projection image data and the second projection image data that are generated by substantially simultaneously causing (a) the first imaging source to emit the first imaging beam and (b) the second imaging source to emit the second imaging beam towards the imager.

3. The method of claim 1, wherein obtaining the first projection image data and the second projection image data comprises:
obtaining the first projection image data and the second projection image data that are generated by sequentially causing (a) the first imaging source to emit the first imaging beam and (b) the second imaging source to emit the second imaging beam towards the imager.

4. The method of claim 1, wherein processing the first projection image data and the second projection image data comprises:
performing template matching to (a) match the first projection image data to one or multiple first template images to determine the first 2D position data and (b) match the second projection image data to one or multiple second template images to determine the second 2D position data, wherein the multiple first template images and the multiple second template images are each associated with at least one part of the target structure.

5. The method of claim 1, wherein processing the first projection image data and the second projection image data comprise:
processing the first projection image data and the second projection image data using an artificial intelligence (AI) engine that is trained to perform 2D position estimation to determine the first 2D position data and the second 2D position data.

6. The method of claim 1, wherein determining the 3D position data comprises one of the following:
performing triangulation to determine the 3D position data based on the first 2D position data and the second 2D position data; and
processing the first 2D position data and the second 2D position data using an Al engine that is trained to determine the 3D position data based on the first 2D position data and the second 2D position data.

7. The method of claim 1, wherein the method further comprises:
obtaining third projection image data that is generated using a third imaging source to emit a third imaging beam towards a third imaging area on the imager to image the target structure from a third angle;
processing the third projection image data to determine third 2D position data associated with the target structure; and
determining the 3D position data associated with the target structure based on the first 2D position data, the second 2D position data and the third 2D position data.

8. A computer system, comprising:
a processor; and
a non-transitory computer-readable medium having stored thereon instructions that, when executed by the processor, cause the processor to perform the following:
obtain first projection image data that is generated using a first imaging source to emit a first imaging beam towards a first imaging area on an imager to image a target structure within a patient from a first angle;
obtain second projection image data that is generated using a second imaging source of the radiation therapy system to emit a second imaging beam towards a second imaging area on the imager to image the target structure from a second angle;
process (a) the first projection image data to determine first two-dimensional (2D) position data associated with the target structure and (b) the second projection image data to determine second 2D position data associated with the target structure; and
based on at least the first 2D position data and the second 2D position data, determine three-dimensional (3D) position data associated with the target structure, thereby tracking the target structure in 3D.

9. The computer system of claim 8, wherein the instructions for obtaining the first projection image data and the second projection image data cause the processor to:
obtain the first projection image data and the second projection image data that are generated by substantially simultaneously causing (a) the first imaging source to emit the first imaging beam and (b) the second imaging source to emit the second imaging beam towards the imager.

10. The computer system of claim 8, wherein the instructions for obtaining the first projection image data and the second projection image data cause the processor to:
obtain the first projection image data and the second projection image data that are generated by sequentially causing (a) the first imaging source to emit the first imaging beam and (b) the second imaging source to emit the second imaging beam towards the imager.

11. The computer system of claim 8, wherein the instructions for processing the first projection image data and the second projection image data and determining the 3D position data cause the processor to:
perform template matching to (a) match the first projection image data to one or multiple first template images to determine the first 2D position data and (b) match the second projection image data to one or multiple second template images to determine the second 2D position data.

12. The computer system of claim 8, wherein the instructions for processing the first projection image data and the second projection image data and determining the 3D position data cause the processor to:
process the first projection image data and the second projection image data using an artificial intelligence (AI) engine that is trained to perform 2D position estimation to determine the first 2D position data and the second 2D position data.

13. The computer system of claim 8, wherein the instructions for determining the 3D position data cause the processor to perform one of the following:
perform triangulation to determine the 3D position data based on the first 2D position data and the second 2D position data; and
process the first 2D position data and the second 2D position data using an Al engine that is trained to determine the 3D position data based on the first 2D position data and the second 2D position data.

14. The computer system of claim 8, wherein the instructions further cause the processor to:
obtain third projection image data that is generated using a third imaging source of the radiation therapy system to emit a third imaging beam towards a third imaging area on the imager to image the target structure from a third angle;
process the third projection image data to determine third 2D position data associated with the target structure; and
determining the 3D position data associated with the target structure based on the first 2D position data, the second 2D position data and the third 2D position data.

15. A radiation therapy system, comprising:
an imager;
a first imaging source to emit a first imaging beam towards a first imaging area on the imager to image a target structure within a patient from a first angle, thereby generating first projection image data;
a second imaging source to emit a second imaging beam towards a second imaging area on the imager to image the target structure from a second angle, thereby generating second projection image data; and
a computer system to (a) obtain the first projection image data and the second projection image data and (b) determine 3D position data associated with the target structure based on the first projection image data and the second projection image data.

16. The radiation therapy system of claim 15, wherein the computer system is to determine 3D position data by performing the following:
process (a) the first projection image data to determine first two-dimensional (2D) position data associated with the target structure and (b) the second projection image data to determine second 2D position data associated with the target structure; and
determine the 3D position data based on (a) the first 2D position data and (b) the second 2D position data.

17. The radiation therapy system of claim 16, wherein the computer system is to determine 3D position data by performing the following:
perform template matching to (a) match the first projection image data to one or multiple first template images to determine the first 2D position data and (b) match the second projection image data to one or multiple second template images to determine the second 2D position data, wherein the multiple first template images and the multiple second template images are each associated with at least one part of the target structure.

18. The radiation therapy system of claim 16, wherein the computer system is to determine 3D position data by performing the following:
process the first projection image data and the second projection image data using an artificial intelligence (AI) engine that is trained to perform one of the following: (a) 2D position estimation to determine the first 2D position data and the second 2D position data and (b) 3D position estimation to determine the 3D position data based on the first projection image data and the second projection image data.

19. The radiation therapy system of claim 15, further comprising:
a third imaging source to emit a third imaging beam towards a third imaging area on the imager to image the target structure from a third angle, thereby generating third projection image data; and
the computer system is further to (a) obtain the third projection image data and the second projection image data and (b) determine the 3D position data associated with the target structure based on the first projection image data, the second projection image data and the third projection image data.

20. The radiation therapy system of claim 19, wherein the computer system is to determine 3D position data by performing one of the following:
perform template matching to (a) match the first projection image data to one or multiple first template images to determine the first 2D position data, (b) match the second projection image data to one or multiple second template images to determine the second 2D position data and (c) match the third projection image data to one or multiple third template images to determine the third 2D position data.

21. The radiation therapy system of claim 19, wherein the computer system is to determine 3D position data by performing one of the following:
process the first projection image data, the second projection image data and the third projection image data using an AI engine that is trained to perform one of the following: (a) 2D position data estimation to determine first 2D position data, second 2D position data and third 2D position data and (b) 3D position data estimation to determine the 3D position data.
